(19) United States
(12) Patent Application Publication
(10) Pub. No.: US 2025/0366594 A1
(43) Pub. Date: Dec. 4, 2025

(54) ADJUSTABLE COSMETIC APPLICATOR HEAD

(71) Applicant: SHENZHEN NUON MEDICAL EQUIPMENT CO.,LTD., Shenzhen (CN)

(72) Inventors: ALAIN DIJKSTRA, Amstelveen (NL); LI XIANG, Shenzhen (CN); Tang Yuanzhou, Shenzhen (CN); Chen Kunjie, Shenzhen (CN)

(73) Assignee: SHENZHEN NUON MEDICAL EQUIPMENT CO., LTD., Shenzhen (CN)

(21) Appl. No.: 19/096,777

(22) Filed: Apr. 1, 2025

(30) Foreign Application Priority Data
Jun. 4, 2024 (CN) 2024212642889
Aug. 26, 2024 (CN) 2024220751731

(51) Int. Cl. A45D 34/04; A46B 7/04; A46B 9/02; A46B 13/02; A46B 13/04; A46B 15/00; A61F 7/00; A61H 15/02; A61N 1/04; A61N 1/32; A61N 5/06; A61N 5/067

(52) U.S. Cl. CPC A45D 34/042; A46B 7/042; A46B 7/046; A46B 9/021; A46B 13/023; A46B 13/04; A46B 15/0022; A46B 15/003; A46B 15/0034; A61F 7/00; A61H 15/02; A61N 1/0472; A61N 1/328; A61N 5/0616; A61N 5/067; A45D 2200/205; A45D 2200/207; A46B 2200/1046; A61F 2007/0052; A61F 2007/0087; A61H 2201/105; A61N 2005/0644; A61N 2005/0651

(57) ABSTRACT

Embodiments of the present invention disclose a cosmetic applicator head, designed for controlled dispensing of cosmetic liquids while integrating stimulation elements for enhanced skin care treatments. The applicator head comprises a main body with a mounting end configured for secure attachment to a cosmetic container, ensuring fluid communication through a liquid discharge channel. The smearing end includes a smearing surface for applying cosmetic liquid, a first surface housing at least one stimulation element, and a second surface featuring multiple convex columns for skin contact, massage, exfoliation, or cleansing. The cosmetic applicator head in some embodiments features a rotatable coupling mechanism between a connecting portion and the main body, enabling adjustable liquid flow regulation.

### TECHNICAL FIELD

The present invention relates to the field of skincare and cosmetic dispensing technology, particularly to multifunctional applicators and liquid supply devices incorporating phototherapy, smart control, and advanced dispensing mechanisms for enhanced cosmetic and skincare treatment.

### BACKGROUND ART

With the continuous advancement of science and technology, people are becoming increasingly conscious of skin maintenance and hygiene, particularly when it comes to facial care. As a result, liquid supply devices for skincare, such as facial cleansing instruments, have been developed to assist in cleansing and massaging the skin, thereby improving cleaning effectiveness and promoting better skincare routines. These devices help remove impurities, excess oil, and cosmetic residues, contributing to healthier skin.

However, despite their benefits, existing skincare liquid supply devices are relatively simple in functionality and often lack the versatility needed to meet the diverse requirements of users. Many conventional devices are designed with limited operational modes, making them insufficient for users who seek a more comprehensive skincare solution. Additionally, factors such as ease of use, convenience, and adaptability to different skincare products remain challenges that impact the overall user experience.

As consumer expectations continue to evolve, there is a growing demand for an improved cosmetic applicator head that not only enhances cleansing efficiency but also offers greater functionality, ease of use, and adaptability. A more advanced solution would address these limitations by integrating innovative features that improve usability, promote hygiene, and provide a more effective and enjoyable skincare experience.

### OBJECTS OF THE INVENTION

Some of the objects of the invention are as follows:

An object of the present invention is to provide a cosmetic applicator head that integrates liquid dispensing, light therapy, and skincare functionalities, improving both cosmetic application and skin treatment in a single device.

Another object of the present invention is to enable precise and customizable liquid flow control through a rotatable connecting portion, allowing users to adjust the dispensing amount based on their needs, reducing waste, and improving adaptability to different skincare routines.

Another object of the present invention is to enhance user convenience and efficiency by integrating light therapy and cleansing functions, allowing simultaneous skin cleaning and skincare treatments.

Another object of the present invention is to improve structural stability and durability by incorporating a sealing gasket, anti-rotation features, and clamping mechanisms, ensuring a secure, leak-proof, and long-lasting design.

Another object of the present invention is to enhance usability by integrating a key unit with an electrical connector and magnet, enabling users to activate functions easily through a simple pressing mechanism.

Another object of the present invention is to integrate electronic components, including a circuit board, battery, phototherapy lamp board, and a vibrator, providing light therapy, vibration massage, and enhanced skincare benefits.

Another object of the present invention is to reduce cleaning difficulty by designing a separate smearing surface and convex massage surface at an obtuse angle, preventing cosmetic buildup and improving hygiene.

Another object of the present invention is to offer a versatile design by incorporating a cleaning brush head with a built-in liquid outlet, making it suitable for deep cleansing, serum application, and cosmetic smearing.

Another object of the present invention is to allow multiple modes of use, including cosmetic application, phototherapy, and massage, either independently or in combination, for a personalized skincare and makeup experience.

Another object of the present invention is to ensure compatibility with various cosmetic containers by designing a mounting end with an adapter, allowing the device to be attached to different skincare and cosmetic bottles.

Another object of the present invention is to improve ergonomic handling and usability by designing the cosmetic applicator head with a detachable and replaceable smearing head, making it adaptable for different cosmetic products and easy to maintain.

### SUMMARY OF THE INVENTION

According to a first aspect of the present invention, a cosmetic applicator head is provided. The cosmetic applicator head comprising: a main body having a mounting end, a smearing end, and a liquid discharge channel extending from the mounting end to the smearing end; at least one stimulation element to provide one or more therapies to a user; wherein the mounting end is configured for attachment to a cosmetic container, ensuring fluid communication between the liquid discharge channel and the cosmetic container; wherein the smearing end comprises a smearing surface through which the liquid discharge channel extends to dispense a cosmetic liquid, a first surface having the at least one stimulation element, and a second surface having multiple convex columns for skin contact.

In one embodiment of the invention, the first surface comprises a light-transmitting area.

In one embodiment of the invention, the convex columns are designed for massaging, exfoliating, or cleansing the skin.

In one embodiment of the invention, the at least one stimulation element is selected from a group consisting of light-emitting diodes (LED), lasers, heating elements, cooling elements, vibration elements, electrodes, micro-current elements, and a combinations thereof.

In one embodiment of the invention, the cosmetic applicator head further comprising a modular mounting system including: a light-shielding piece positioned within the main body to direct emitted light through the light-transmitting area; and a light- transmitting cover secured over the light-shielding piece.

In one embodiment of the invention, the light-shielding piece and light-transmitting cover are secured via a snap-fit mechanism or bonding.

In one embodiment of the invention, the main body comprises a shell and a brush head, the shell including a smearing surface and a mounting groove positioned on one side of the smearing surface; wherein the brush head comprises a second surface, a boss, and a plug-in portion extending from the second surface, the plug-in portion being configured to be inserted into the mounting groove for secure attachment.

In one embodiment of the invention, the first and second surfaces are positioned at an obtuse angle relative to the smearing surface.

In one embodiment of the invention, the liquid discharge channel comprises: a first channel within the accessory body; and a second channel within a liquid guide pipe, with a sealing ring ensuring a secure fit between the two channels.

In one embodiment of the invention, the cosmetic applicator head further comprising an adapter at the mounting end, configured to accommodate cosmetic containers of varying sizes.

According to a second aspect of the present invention, a cosmetic applicator head is provided. The cosmetic applicator head comprising: a main body including a storage cavity and a liquid outlet; at least one stimulation element to provide therapy to a user; a connecting portion having a liquid channel, wherein one end of the liquid channel is connected to the liquid outlet, and the other end is configured for attachment to a liquid container; a rotatable coupling between the connecting portion and the main body, enabling alignment adjustment between the liquid channel and the liquid outlet to regulate liquid flow.

In one embodiment of the invention, the liquid outlet is positioned on a surface that includes brush bristles for enhanced cleansing functionality.

In one embodiment of the invention, the connecting portion comprises: a base; a connecting protrusion extending from the base, wherein the liquid channel passes through the connecting protrusion; and wherein the connecting protrusion is rotatably matched with the rotating hole in the main body, enabling controlled alignment between the liquid channel and the liquid outlet.

In one embodiment of the invention, the rotatable coupling allows the liquid channel to be positioned in a fully aligned state for maximum liquid discharge, a fully misaligned state to block liquid flow and intermediate positions for controlled dispensing.

In one embodiment of the present invention, the cosmetic applicator head further comprising a sealing gasket positioned between the connecting component and the main body to prevent liquid leakage.

In one embodiment of the present invention, the main body comprises an outer shell and an inner support, wherein the inner support incorporates a connecting channel that links the liquid channel to the liquid outlet.

In one embodiment of the invention, the inner support includes a rotating hole accommodating the connecting protrusion, allowing the connecting component to pivot for liquid flow control.

In one embodiment of the invention, the cosmetic applicator head further comprising a limiting protrusion on the main body and a first-stop protrusion on the connecting component to restrict rotational movement within a predetermined range.

In one embodiment of the invention, the connecting component includes a second stop protrusion positioned along the rotation path to provide tactile feedback when adjusting liquid flow.

In one embodiment of the invention, the at least one stimulation element is selected from a group consisting of light-emitting diodes (LED), lasers, heating elements, cooling elements, vibration elements, electrodes, micro-current elements, and combinations thereof.

In one embodiment of the invention, the brush bristles are detachable and secured using a buckle mechanism, screws, or a magnetic attachment.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, and IR frequencies and wavelengths. Preferably, it refers to low-level electromagnetic radiation of low-level red and near-infrared (NIR) light. It is to be noted here that IR radiation can be categorized into several categories according to respective wavelength ranges which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm). In this regard, light application is at relatively low energy densities, typically below about 500 mW, as compared to other forms of laser therapy that are used for ablation, cutting, and thermally coagulating tissue. In some instances, electromagnetic radiation can also be in wavelengths in the blue or ultraviolet regions, especially for treatment of conditions that occur at the skin surface, such as psoriasis or infection.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other component on the piece.

In the context of the specification, the terms “first’, “second” and “third” are only used for descriptive purposes and do not implicate the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

In the context of the specification, the term “smearing surface” refers to the part of the applicator that comes into direct contact with the cosmetic liquid and facilitates its application onto the user's skin. The smearing surface may be smooth, textured, or include additional features such as convex columns for enhanced application effects.

In the context of the specification, the term “phototherapy component” refers to an assembly comprising one or more light-emitting elements, a control circuit, and an optional power source, configured to emit electromagnetic radiation for skincare treatments.

In the context of the specification, the term “convex columns” refers to raised protrusions on the smearing surface, which may serve functions such as massaging, exfoliating, or evenly distributing cosmetic products during application.

In the context of the specification, the term “light-transmitting area” refers to a section of the applicator that allows light emitted by the phototherapy component to pass through without significant obstruction. This area may be made of transparent or translucent materials to ensure effective light exposure.

In the context of the specification, the term “sealing ring” refers to an elastic component that ensures a leak-proof connection between the applicator and the cosmetic container, preventing unwanted leakage of liquid cosmetics.

In the context of the specification, the term “light-shielding piece” refers to a structural component designed to control the emission of light from the phototherapy component, ensuring that light is directed appropriately for its intended therapeutic effects.

In the context of the specification, a sealing gasket refers to a component positioned between structural parts to prevent leakage and minimize friction between harder materials.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1 is a schematic diagram illustrating the structure of a cosmetic applicator head, in accordance with an embodiment of the present invention.

FIG. 2 is an exploded view of a cosmetic applicator head, in accordance with an embodiment of the present invention.

FIG. 3 shows an enlarged view of section A in FIG. 2 showing detailed structure feature, in accordance with an embodiment of the present invention.

FIG. 4 shows a cross-sectional view of the cosmetic applicator head, in accordance with an embodiment of the present invention.

FIG. 5 shows an enlarged view of section B in FIG. 4 showing internal components, in accordance with an embodiment of the present invention.

FIG. 6 shows the structure of a translucent cover shown in FIG. 2, in accordance with an embodiment of the present invention.

FIG. 7 shows the cosmetic applicator head with the protective cover, in accordance with an embodiment of the present invention.

FIG. 8 is a schematic diagram illustrating the assembly structure of a cosmetic applicator head, in accordance with an alternate embodiment of the present invention.

FIG. 9 is an exploded view showing the structural components of the cosmetic applicator head of FIG. 8, in accordance with an embodiment of the present invention.

FIG. 10 shows the internal support structure of the cosmetic applicator head, in accordance with an embodiment of the present invention.

FIG. 11 shows a cross-sectional view of the cosmetic applicator head, in accordance with an embodiment of the present invention.

FIG. 12 shows a bottom cross-sectional view of the cosmetic applicator head, in accordance with an embodiment of the present invention.

FIG. 13 shows the structure of a gasket integrated with the cosmetic applicator head, in accordance with an embodiment of the present invention.

FIG. 14 shows a bottom view of the main body of the cosmetic applicator head, in accordance with an embodiment of the present invention.

FIG. 15 shows a connecting part associated with the cosmetic applicator head, in accordance with an embodiment of the present invention.

FIG. 16 shows a cross-sectional view of the connecting part of the cosmetic applicator head, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signifies the presence of a component.

Embodiments of the present invention disclose a cosmetic applicator head. The cosmetic applicator head comprises a main body with a mounting end and a smearing end, connected by a liquid outlet channel. The mounting end is designed to attach to a cosmetic container, allowing the liquid to flow through the channel to the smearing surface. The smearing end consists of a smearing surface, a first surface, and a second surface. The first surface features a light-transmitting area, and the second surface is equipped with multiple convex columns to enhance the smearing effect. The cosmetic applicator head comprises at least one stimulation element or a combination of stimulation elements embedded within the main body. The stimulation element is selected from a group consisting of light-emitting diodes (LEDs), lasers, heating elements, cooling elements, vibration elements, micro-current elements, and combinations thereof. The stimulation element can be a phototherapy assembly embedded within the main body and positioned in alignment with the light-transmitting area, providing therapeutic light treatment during cosmetic application.

The main body includes a shell and a light-shielding piece, the light-shielding piece is a cylindrical component with an open end and a closed baffle containing a light-transmitting hole. The light-shielding piece is securely embedded in the shell using buckles that snap into corresponding grooves, ensuring a stable structure. For liquid dispensing, the main body incorporates a liquid guide pipe housed within a mounting bracket, forming a two-part liquid outlet channel. A sealing ring is positioned within a jack to prevent leaks, and an adapter at the mounting end allows secure attachment to various cosmetic containers. A brush head is attached to the shell via a plug-in portion that fits into a mounting groove. The brush head may be secured through over-injection molding, and the height of the convex bosses on its second surface gradually increases from the perimeter to the center, optimizing the cosmetic application.

In another embodiment of the present invention, the cosmetic applicator head integrates liquid dispensing and phototherapy functions. The main body of the cosmetic applicator head features a storage cavity that holds liquid, a liquid outlet, and a light-transmitting area. The cosmetic applicator head comprises at least one stimulation element or a combination of stimulation elements embedded within the main body. The stimulation element is selected from a group consisting of light-emitting diodes (LEDs), lasers, heating elements, cooling elements, vibration elements, micro-current elements, and combinations thereof. The stimulation element can be a phototherapy lamp panel placed within the storage cavity, directing therapeutic light toward the light-transmitting area. The connecting portion of the device includes a liquid channel, where one end connects to the liquid outlet while the other end attaches to a liquid container. The rotatable design of the connecting portion allows users to adjust the liquid flow by varying the communication area between the liquid channel and the outlet.

The main body consists of a shell and an inner support, where the inner support is fixed within the storage cavity and provides a rotating hole and a connecting channel to control liquid flow. The connecting portion is equipped with a base and a protruding convex section that fits into the rotating hole, enabling a rotary adjustment mechanism. A sealing gasket is positioned between the base and the inner support, preventing leaks while ensuring smooth liquid flow through a precisely aligned connecting port. Additionally, an anti-rotation mechanism comprising an anti-rotation protrusion on the inner support and a corresponding groove on the sealing gasket that prevents misalignment during rotation.

Several additional functionalities may also be added to the cosmetic applicator head. For example, the cosmetic applicator head may have at least one additional stimulation element to impart additional therapy to the user. The at least one stimulation element is selected from the group consisting of lasers, heating elements, cooling elements, vibration elements, electrodes, and micro-current elements. The vibration element can be used for massaging the face, or the micro-current element is used to impart micro-current to the skin of the user. The cosmetic applicator head may have one or more sensors to sense the one or more parameters of the skin of the user. Moreover, activation and control of specific stimulation elements may be based upon feedback received from the one or more sensors. The cosmetic applicator may comprise a controller connected to an external device to control the functioning of the stimulation elements in the cosmetic applicator head.

Several embodiments of the present invention will now be described in detail with reference to FIGS.

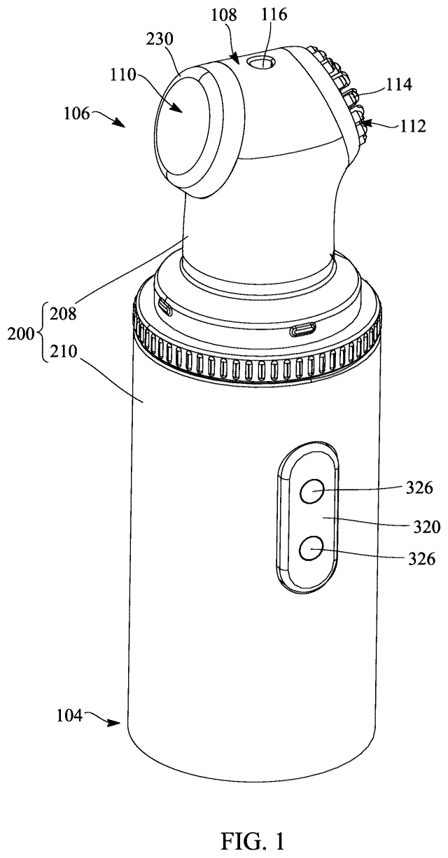

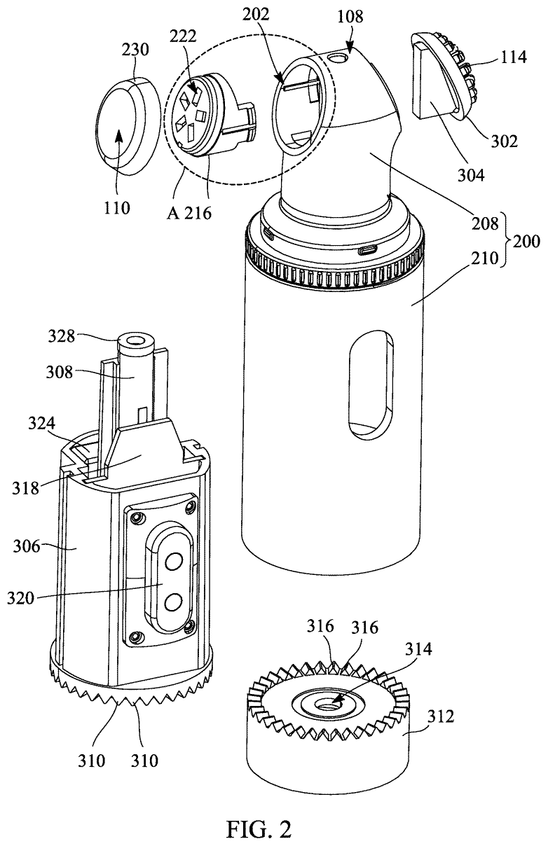

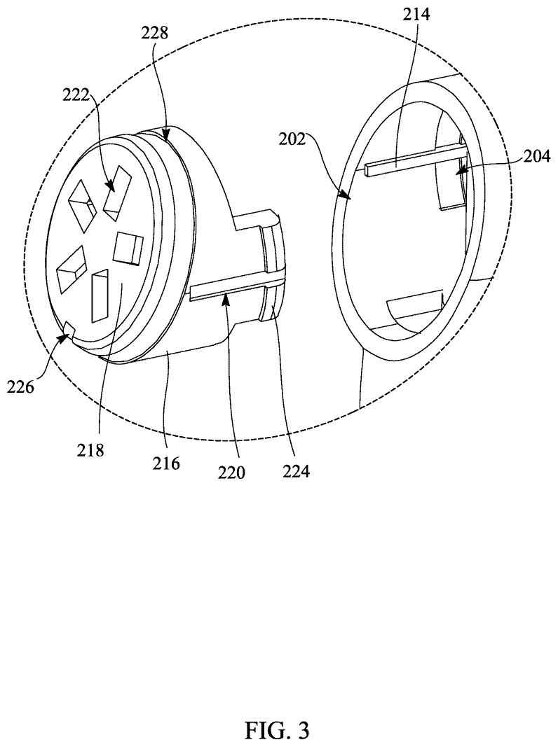

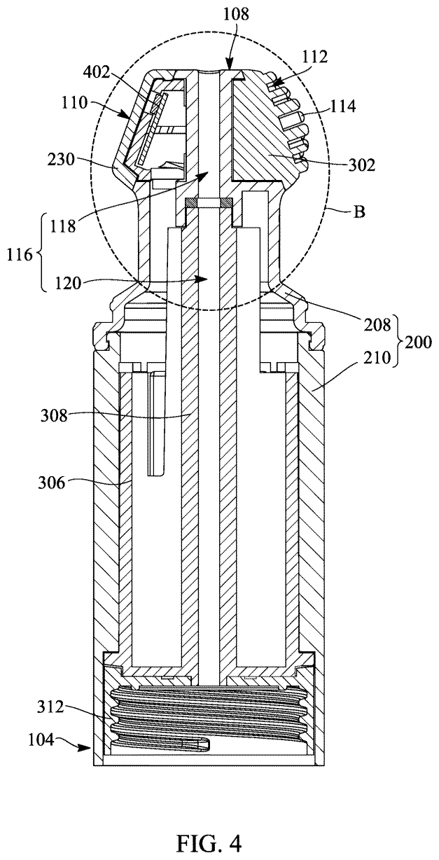

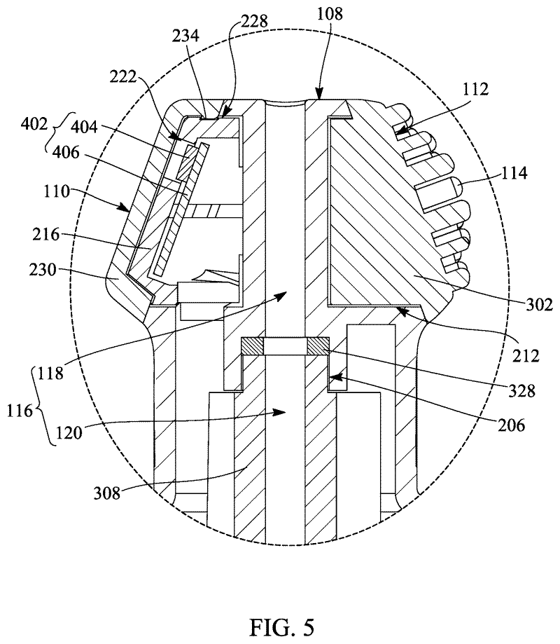

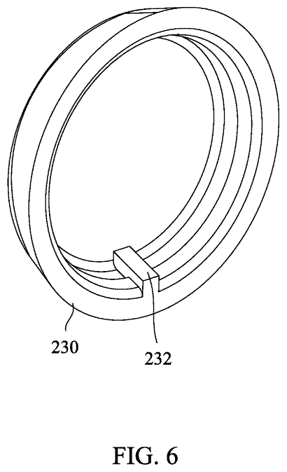

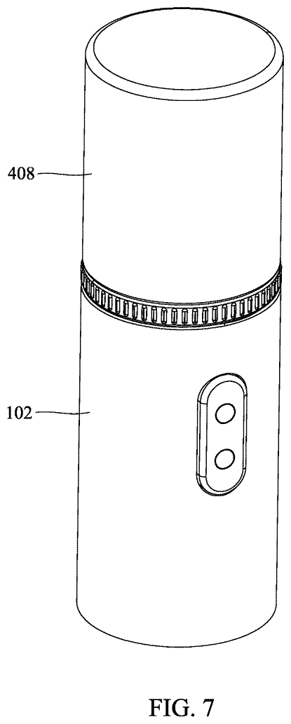

Referring to FIG. 1 to FIG. 7, the cosmetic applicator head comprises an accessory body 102 and a phototherapy component 402. The accessory body 102 features a mounting end 104, a smearing end 106, and a liquid discharge channel 116 that extends from the mounting end 104 to the smearing end 106. The mounting end 104 is designed for attachment to the container mouth of a cosmetic container, ensuring that the liquid discharge channel 116 remains in fluid communication with the cosmetic container. Additionally, the applicator may include smart connectivity features such as Bluetooth or Wi-Fi, enabling users to control phototherapy settings via a mobile app. This allows users to select specific wavelengths, monitor usage statistics, and receive personalized skincare recommendations.

The smearing end 106 is configured with a smearing surface 108, a first surface 110, and a second surface 112. The liquid discharge channel 116 extends through the smearing surface 108, allowing the cosmetic liquid to be dispensed directly onto it. The first surface 110 and the second surface 112 are positioned at angled orientations relative to the smearing surface 108. Additionally, the first surface 110 includes a light-transmitting area, while the second surface 112 is equipped with multiple convex columns 114 to enhance its functionality.

The phototherapy component 402 is integrated within the accessory body 102 and is strategically aligned with the light-transmitting area on the first surface 110. Specifically, the phototherapy component 402 comprises at least one light-emitting element 404, which is designed to emit light of specific wavelengths. When in use, the light emitted by the light-emitting element 404 passes through the translucent area and irradiates the skin, providing therapeutic benefits. The phototherapy component 402 may utilize one or more types of light, including red light, blue light, violet light, or other wavelengths, depending on the intended treatment purpose. Different configurations of light-emitting elements 404 can be incorporated as required. In addition to traditional phototherapy, the system may incorporate multi-mode phototherapy with automatic skin condition detection, adjusting the wavelength and intensity accordingly.

The power supply for the phototherapy component 402 can be provided through a battery 324 housed within the cosmetic smear accessory or via an external power source, such as a mobile power bank. To further improve user comfort, the applicator may include a temperature control mechanism featuring thermoelectric components, allowing users to activate a heating or cooling function. This feature enhances cosmetic absorption and provides a soothing skincare experience.

When the accessory body 102 is affixed to the container mouth of a cosmetic container via the mounting end 104, the liquid discharge channel 116 establishes a direct flow path between the inner cavity of the cosmetic container and the smearing surface 108. This mechanism allows the cosmetic liquid to be dispensed smoothly onto the smearing surface 108, facilitating easy application onto the skin. Furthermore, a smart dispensing mechanism may be integrated, incorporating a pressure-sensitive liquid dispensing system that adjusts the amount of cosmetic liquid based on skin contact pressure. A micro-pump can be employed for controlled dispensing, ensuring reduced waste and optimal application.

The first surface 110 and the second surface 112 are positioned on opposite sides of the smearing surface 108 and can be arranged symmetrically or asymmetrically. Both surfaces extend at an obtuse angle relative to the smearing surface 108, gradually sloping away in the direction of the mounting end 104. This ergonomic design ensures comfortable handling of the accessory body 102 or the cosmetic container, allowing the convex columns 114 on the first surface 110 and second surface 112 to make effective contact with the skin for improved usability. An improved ergonomic grip with pressure-sensitive controls can be integrated, allowing users to adjust phototherapy settings and liquid dispensing intuitively during application.

Furthermore, the convex columns 114 are designed with smooth, curved ends and can be made from either hard or soft materials, depending on the intended application. During use, these convex column 114 can be utilized to gently rub against the skin, offering additional functions such as cleansing, massaging, or exfoliating, thereby enhancing the overall cosmetic application experience. To maintain hygiene, the accessory can be equipped with a self-cleaning UV sterilization system that sanitizes the smearing surface and convex columns after each use. Additionally, replaceable applicator heads with antibacterial coatings can be provided to further enhance hygiene and longevity.

The liquid discharge channel 116 extends to the smearing end 106 by incorporating a relative mounting end 104 and a smearing end 106 within the accessory body 102. The smearing surface 108 is flanked by the first surface 110 and a second surface 112, with the liquid discharge channel 116 passing through the smearing surface 108. The first surface 110 and the second surface 112 are positioned at specific angles relative to the smearing surface 108. Additionally, the first surface 110 includes a light-transmitting area, housing a phototherapy component 402 within the accessory body 102, while the second surface 112 is embedded with multiple convex columns 114.

During use, the user has the flexibility to apply cosmetics using the smearing surface 108 alone, activate the phototherapy component 402 for light-based therapy, use the convex columns 114 for skin cleansing or massage, or combine multiple functionalities by first applying cosmetics and then utilizing phototherapy. The design ensures that the second surface 112 and the smearing surface 108 are arranged at an obtuse angle, keeping the smearing surface 108 separate from the convex columns 114. This not only allows independent use of each function but also enhances user convenience by preventing cosmetic buildup between the convex columns 114, thereby simplifying cleaning and improving the overall user experience.

In certain embodiments, the accessory body 102 consists of a shell 200 and a light-shielding piece 216. The shell 200 incorporates the smearing surface 108 and the first surface 110, while the light-shielding piece 216 is a cylindrical structure with one open end and a closed end sealed by a baffle plate 218. The baffle plate 218 features a light-transmitting hole 222 that aligns with the first surface 110, allowing light from the phototherapy component 402 to be emitted externally.

The phototherapy component 402 includes a circuit board 406 with one or more light-emitting elements 404. The circuit board 406 is housed within the light-shielding piece 216, with the light-emitting elements 404 positioned in alignment with the light-transmitting hole 222. When multiple light-emitting elements 404 are incorporated, the baffle plate 218 is equipped with corresponding light-transmitting holes 222 for each element, ensuring focused light emission and enhanced therapeutic effect. The light-shielding piece 216 is securely affixed to the shell 200 through bonding or a snap-fit mechanism.

In some embodiments, the shell 200 integrates a housing and a light-transmitting cover 230, with the light-shielding piece 216 being either bonded or snap-fitted to one of these components. This configuration ensures effective light transmission while maintaining the structural integrity of the device.

In some embodiments, the shell 200 features a smearing surface 108 and a mounting port 202 located on one side of the smearing surface 108. The light-shielding piece 216 is installed within the mounting port 202, while the light-transmitting cover 230 encloses the mounting port 202 and forms the first surface 110. This configuration ensures that the light-transmitting cover 230 is offset from the surface of the light-shielding piece 216, creating the first surface 110. The shell 200, light-shielding piece 216, and light-transmitting cover 230 can be separately molded to simplify the manufacturing process. During assembly, the light-shielding piece 216 is inserted into the shell 200 through the mounting port 202, and the light-transmitting cover 230 is secured over the port, effectively reducing the complexity of the shell's formation. Additionally, the phototherapy component 402 can be installed through the mounting port 202, allowing for more convenient assembly. In alternative embodiments, the section comprising the smearing surface 108, the first surface 110, and the second surface 112 may be integrally molded as a single unit.

To facilitate secure installation, the light-shielding piece 216 is equipped with multiple buckles 224, which correspond to buckle grooves 204 on the inner wall of the shell 200. During installation, the buckles 224 snap into the buckle grooves 204, enabling a straightforward and stable attachment by simply inserting the light-shielding piece 216 into position.

In some embodiments, either the outer perimeter of the light-shielding piece 216 or the inner wall of the shell 200 is equipped with a guide rib 214, while the other component features a corresponding guide groove 220. The guide groove 220 runs along the opening direction of the mounting port 202, ensuring the correct positioning of the light-shielding piece 216 and preventing misalignment during assembly. Optionally, the inner wall of the shell 200 may feature two guide ribs 214, arranged opposite to each other, while the light- shielding piece 216 is equipped with two matching guide grooves 220 on opposing sides. These guide ribs 214 and the guide grooves 220 align in a one-to-one configuration, ensuring a precise and secure fit during installation.

In some embodiments, either the light-transmitting cover 230 or the light-shielding piece 216 is equipped with a positioning rib 232, while the other features a corresponding positioning groove 226. This design facilitates the precise alignment and assembly of the light-transmitting cover 230 while preventing misalignment during installation.

Additionally, in some embodiments, either the light-transmitting cover 230 or the light-shielding piece 216 includes a clasp 234, with the other component featuring a corresponding card groove 228. During assembly, the light-transmitting cover 230 is simply secured onto the light-shielding piece 216 until the clasp 234 locks into the card groove 228, ensuring a firm and convenient attachment.

In certain embodiments, the accessory body 102 consists of a shell 200 and a brush head 302. The shell 200 includes a smearing surface 108 and a mounting groove 212 positioned on one side of the smearing surface. The brush head 302 is inserted into the mounting groove 212 and consists of the second surface 112, multiple convex columns 114, and a plugging portion 304 that extends from the second surface 112. The plugging portion 304 is securely fitted into the mounting groove 212, allowing the shell 200 and brush head 302 to be manufactured separately before being assembled, fixed, or over-molded. This flexibility enables the selection of different materials for the convex columns 114 and brush head 302, allowing for customization based on production requirements. The convex columns 114 and brush head 302 may be made from silicone or hard plastic as a single unit.

Furthermore, in some embodiments, the height of the protrusions on the convex column 114 gradually increases from the outer edges toward the center of the second surface 112. This means that the convex columns 114 are positioned lower at the periphery and higher in the center, allowing them to better conform to contoured areas of the skin, such as the eye region, enhancing the cleansing and massaging effect in those areas.

Optionally, the shell 200 and brush head 302 can be over-molded, meaning that one of these components is molded first, and the other is then injection-molded around it as an insert. This process ensures a secure and durable attachment of the brush head 302 to the shell 200.

In another embodiment, the accessory body 102 consists of a shell 200 and a mounting bracket 306. The shell 200 features a smearing surface 108 and a mounting end 104, while the mounting bracket 306 is housed within the shell 200 and includes a liquid guide pipe 308. The liquid discharge channel 116 is divided into a first channel 118 within the shell 200 and a second channel 120 within the liquid guide pipe 308. One end of the first channel 118 extends through the smearing surface 108, while the other end connects to the second channel 120.

By forming the liquid discharge channel 116 through the combination of the first channel 118 in the shell 200 and the second channel 120 in the liquid guide pipe 308, the overall structure of the shell 200 is simplified, and manufacturing complexity is reduced.

In some embodiments, the accessory body 102 includes a sealing ring 328. The shell 200 has a jack 206, with one end of the first channel 118, opposite the smearing surface 108, penetrating through the bottom wall of the jack 206. Meanwhile, one end of the liquid guide pipe 308, opposite the mounting end 104, is inserted into the jack 206, with the sealing ring 328 positioned within the jack 206, ensuring a secure fit between the end face of the liquid guide pipe 308 and the bottom wall of the jack 206. During assembly, the sealing ring 328 is first placed inside the jack 206, followed by the insertion of the liquid guide pipe 308. Once in place, the sealing ring 328 enhances the sealing properties, simplifies installation, and allows for a reduction in the radial size of the jack 206.

In some embodiments, the accessory body 102 further includes an adapter 312, with the mounting end 104 designed as an open opening. The adapter 312 is installed in the mounting end 104 and features a hole 314 aligned with the second channel 120. The adapter 312 serves as a connection point for attaching the container mouth of a cosmetic container. The design allows for compatibility with cosmetic containers of various sizes, requiring only modifications to the adapter 312 mold, rather than the entire shell 200, effectively reducing production costs. Alternatively, the shell 200 and the adapter 312 can be integrally molded, eliminating the need for a separate adapter 312.

In some embodiments, the adapter 312 has an open end opposite the hole 314, with an internal thread on its inner wall. This internal thread allows the adapter 312 to be screwed onto the container mouth of a cosmetic container, making installation efficient and compatible with existing containers. Alternatively, a convex part may be positioned at the mounting end 104, allowing the container mouth to be plugged into the convex part, ensuring a secure seal.

In some embodiments, either the mounting bracket 306 or the adapter 312 is equipped with multiple first clamping teeth 310, while the other features at least one second clamping tooth 316. The first clamping teeth 310 are evenly spaced around the circumference of the second channel 120, with each second clamping tooth 316 fitting between two adjacent first clamping teeth 310. Since some cosmetic containers, such as eye cream bottles, have asymmetrical or non-circular shapes (e.g., flat designs), this setup allows for precise alignment before final assembly.

To ensure correct positioning, the cosmetic container is first threaded onto the adapter 312, which is then inserted into the mounting end 104. The adapter 312 or shell 200 is rotated until the container aligns optimally with the first surface 110 and the second surface 112 of the smearing end 106, preventing further movement. Finally, the adapter 312 is secured to the shell 200 using glue or screws, ensuring that each time the user attaches the cosmetic application accessory to a cosmetic container, it remains positioned for easy handling, phototherapy, and cleansing/massage, enhancing the user experience.

In some embodiments, the accessory body 102 includes a main control board 318 and a battery 324, both electrically connected to the phototherapy component 402. The battery 324 and the main control board 318 are positioned on opposite sides of the liquid guide pipe 308, extending along its length in a compact, elongated form. This design optimizes space utilization within the shell 200, reducing the accessory's overall radial size and making it more compatible with small cosmetic containers, such as eye cream bottles.

In some embodiments, the main control board 318 is equipped with a key 320. A through-hole is positioned on the shell 200 corresponding to the key 320, allowing it to be exposed. Optionally, the keycaps of the key 320 feature two conductive parts 326, which are electrically connected to the charging circuit on the main control board 318. These conductive parts 326 enable the battery 324 to be charged via a charger connection.

In some embodiments, the shell 200 consists of an upper shell 208 and a lower shell 210. The lower shell 210 is a cylindrical structure with open ends, forming the mounting end 104 at its lower end. The upper shell 208 is attached to the upper end of the lower shell 210, with its far end forming the smearing end 106. During assembly, the mounting bracket 306, battery 324, and main control board 318 are first installed in the lower shell 210. The upper shell 208 is then attached to the lower shell 210, simplifying the assembly process.

In some embodiments, the cosmetic application fitting includes a detachable protective cover 408, which covers at least the smearing end 106. This design protects the smearing end 106 from contamination when not in use.

In another embodiment of the present invention, the liquid outlet hole of the cosmetic applicator head is provided on the surface having brush bristles, and the flow of liquid from a cosmetic bottle is controlled by providing a connecting portion.

In an embodiment of the present invention, the cosmetic applicator head comprises one or more stimulation elements in the accessory body 102 to provide additional therapy to the user. The one or more stimulation elements are selected from a group consisting of lasers, heating elements, cooling elements, vibration elements, electrodes, micro-current elements, and a combination thereof. The micro-current element provides micro-current therapy to the user, the vibration element provides massage therapy and the heating element provides heat therapy. The micro-current element or electrodes are either provided on the first surface or the second surface.

In an embodiment of the present invention, the cosmetic applicator head further comprises one or more sensors to sense one or more parameters of the skin. The cosmetic applicator head may communicate one or more parameters of the skin to the external communication device. The one or more sensors upon sensing the one or more parameters of the skin may regulate the operation of one or more stimulation elements.

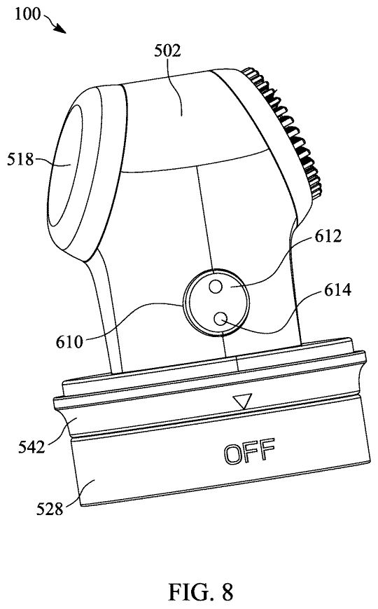

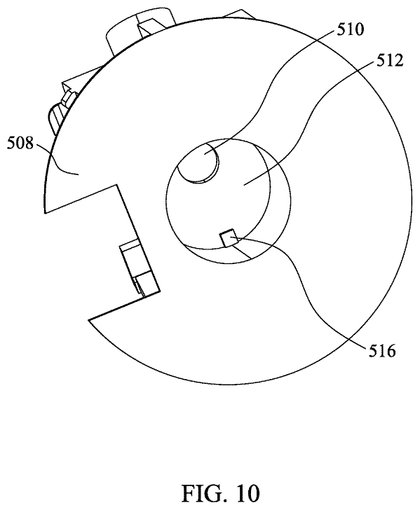

Referring to FIG. 8 to FIG. 11, a schematic representation of the assembly structure of the cosmetic applicator head is shown. The cosmetic applicator head 100 comprises a main body 502, a phototherapy lamp board 526, and a connecting portion 528. The main body 502 features a storage cavity 506 and a liquid outlet 524 that establishes communication with the storage cavity 506. Additionally, a designated section of the main body 502 is configured as a light-transmitting area 518. The phototherapy lamp board 526 is positioned within the storage cavity 506, directly facing the light-transmitting area 518 to ensure effective light transmission. The connecting portion 528 includes a liquid channel 534, with one end connecting to the liquid outlet 524 and the other end designed for attachment to a liquid container. The connecting portion 528 is rotatably linked to the main body 502, enabling adjustment of the liquid channel 534′s alignment with the liquid outlet 524 to regulate liquid flow.

The main body 502 serves as the core operational unit of the cosmetic applicator head 100, integrating light therapy and cleansing functionalities. The inclusion of the light-transmitting area 518 allows the phototherapy function to be realized by permitting light passage. This light-transmitting area 518 may be implemented as a separate light outlet cover, an opening, or a thin-walled section, whereas the remaining portion of the main body 502 maintains an opaque structure. Additionally, the device may incorporate smart connectivity features, including Bluetooth or Wi-Fi, enabling users to control the phototherapy settings via a mobile application. Through the app, users can select specific light wavelengths, track usage statistics, and receive personalized skincare recommendations. An intelligent skin analysis system can be integrated to automatically adjust the light intensity and wavelength based on real-time skin condition detection, ensuring optimal treatment.

Positioned within the storage cavity 506, the phototherapy lamp board 526 is oriented toward the light-transmitting area 518, ensuring that phototherapy light effectively reaches the target area for treatment. The phototherapy functionality supports multiple wavelengths, including red, blue, green, and near-infrared (NIR) light. NIR light, in particular, enables deeper penetration, promoting collagen production and improving skin elasticity. The integration of automatic intensity adjustments based on detected skin conditions enhances treatment effectiveness.

The connecting portion 528, equipped with a liquid channel 534, enables fluid communication between the liquid outlet 524 and an external liquid container. By rotating the connecting portion 528, the liquid flow rate can be precisely adjusted, thereby enhancing user convenience and operational control. Additionally, the liquid dispensing system is upgraded with a pressure-sensitive mechanism that adjusts the liquid flow based on skin contact pressure, preventing wastage and ensuring even application. A micro-pump system is integrated to regulate liquid dispensing, allowing precise and consistent application of skincare products.

The adjustable communication area between the liquid channel 534 and the liquid outlet 524 permits varying degrees of liquid discharge. This includes a fully misaligned position where no liquid flows, a fully aligned position enabling maximum liquid discharge, and intermediate positions allowing partial liquid flow for controlled dispensing. The liquid container may hold various substances such as facial cleansers, shower gels, or skincare essences, offering users flexibility in product selection. The structure of the liquid supply head is further enhanced to support an adjustable mounting system, enabling compatibility with various skincare product containers.

The design effectively integrates the liquid supply function with phototherapy, expanding the functionality and application range of the cosmetic applicator head 100. The adjustable liquid channel 534 facilitates precise control over liquid flow, improving usability. The rotatable connection between the main body 502 and the connecting portion 528 enhances user convenience by simplifying liquid flow adjustments.

Structurally, the main body 502 consists of an outer shell 504 and an inner support 508. The outer shell 504 encloses the storage cavity 506 and the liquid outlet 524, while the inner support 508, fixed within the storage cavity 506, provides structural integrity. The inner support 508 incorporates a rotating hole 512 and a connecting channel 510, with one end linked to the rotating hole 512 and the other end connected to the liquid outlet 524. Additionally, the inner support 508 features a liquid outlet hole 514 for dispensing liquid. A temperature control mechanism is integrated within the main body 502, using thermoelectric components for warming or cooling functions. This feature improves skincare product absorption and enhances user comfort. Temperature adjustments can be made through the mobile app or via a manual interface on the device.

The outer shell 504 forms the external framework of the cosmetic applicator head 100, proportionally sized to fit the inner support 508 while enhancing overall stability and aesthetics. The fixed positioning of the inner support 508 prevents component displacement within the storage cavity 506, ensuring durability and ease of maintenance. The separation of the inner support 508 from the outer shell 504 also simplifies cleaning and reduces manufacturing complexity. To maintain hygiene, a UV sterilization system is incorporated to sanitize the dispensing surface and phototherapy area after each use. Replaceable applicator heads with antibacterial coatings further reduce bacterial contamination and enhance user safety.

The rotating hole 512 in the inner support 508 allows the connecting portion 528 to pivot, thereby modifying the communication area between the connecting channel 510 and the liquid outlet 524 for precise liquid flow regulation. This design eliminates the need for disassembly or reassembly when adjusting liquid flow.

Furthermore, the main body 502 includes a connecting ring 542, with the lower end of the outer shell 504 affixed to it. The base 530 is encased within the connecting ring 542 through a sealing ring 602, allowing the base 530 to rotate relative to the sealing ring 602 and the connecting ring 542, ensuring smooth and controlled liquid dispensing. To improve power efficiency, the cosmetic applicator head 100 supports wireless charging and external power connectivity, such as USB-C or a magnetic charging dock, offering convenient and flexible recharging options.

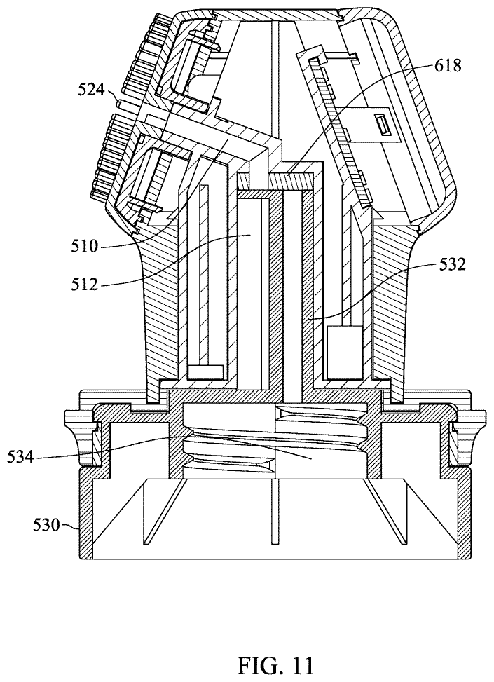

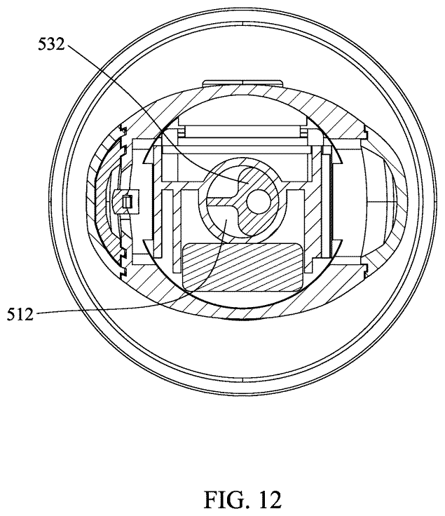

Referring to FIG. 11 and FIG. 12, the connecting portion 528 consists of a base 530 and a connecting protrusion 532 extending from the base 530. The liquid channel 534 runs through the connecting protrusion 532, which is designed to fit rotatably within the rotating hole 512. This rotational fit allows for the adjustment of the communicating area between the liquid channel 534 and the connecting channel 510, thereby regulating the liquid flow.

The base 530 serves as the foundational structure of the connecting portion 528, providing stable support. The connecting protrusion 532, extending outward from the base 530, is the key structural component through which the liquid channel 534 passes. The connecting protrusion 532 is inserted into the rotating hole 512 of the inner support 508, ensuring a secure and functional rotational mechanism for controlled liquid flow.

As the liquid passes from the container, it moves sequentially through the liquid channel 534 in the connecting protrusion 532, then into the connecting channel 510, and finally reaches the liquid outlet 524. By rotating the connecting protrusion 532, the degree of communication between the liquid channel 534 and the connecting channel 510 can be altered, enabling precise regulation of liquid discharge.

To optimize rotation and reduce friction, at least part of the connecting protrusion 532 features a non-circular design, while the rotating hole 512 maintains a circular shape. This creates a structured gap between the non-circular portion of the connecting protrusion 532 and the inner wall of the rotating hole 512, thereby minimizing rotational resistance. In particular, the connecting protrusion 532 may have a partially circular circumference, while the remaining portion includes a concave section to further facilitate smooth rotation.

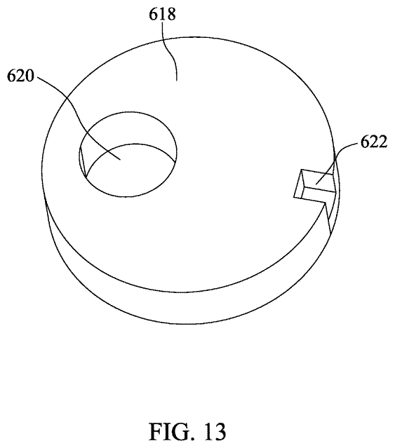

Referring to FIG. 11 and FIG. 13, the cosmetic applicator head 100 further includes a sealing gasket 618, which is positioned between one end of the base 530 that is opposite to the connecting protrusion 532 and the inner support 508. The sealing gasket 618 features a connecting port 620 aligned with the connecting channel 510, allowing the liquid channel 534 to establish communication with the connecting channel 510 through the connecting port 620.

The placement of the sealing gasket 618 within the rotating hole 512 effectively seals the gap between the top hole wall of the connecting protrusion 532 and the rotating hole 512, preventing any potential leakage. Additionally, since the connecting protrusion 532 makes contact with the inner support 508 via the sealing gasket 618, the softer material of the gasket acts as a buffer. This design minimizes direct friction between the harder structural components, thereby enhancing the durability and longevity of the cosmetic applicator head 100.

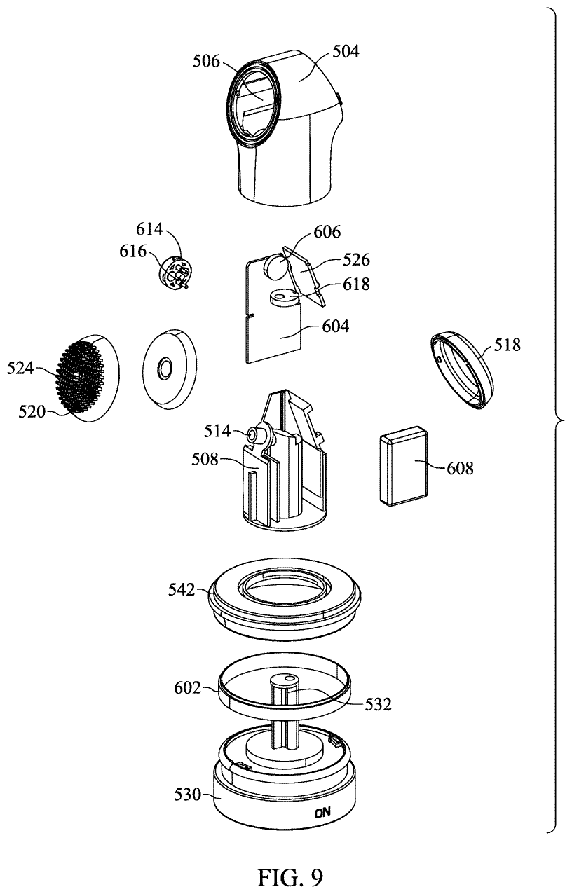

Referring to FIGS. 9, 10, and 13, the inner support 508 is equipped with an anti- rotation protrusion 516, while the sealing gasket 618 features a corresponding anti-rotation groove 622. This interlocking design ensures that the sealing gasket 618 remains securely in place and is not inadvertently rotated by the movement of the connecting portion 528.

Additionally, as illustrated in FIG. 9, the cosmetic applicator head 100 further incorporates a circuit board 604 and a battery 608. The circuit board 604, battery 608, and phototherapy lamp board 526 are electrically connected and securely housed within the inner support 508. The circuit board 604 is also equipped with a vibrator 606. The power system is designed to efficiently support all integrated smart functionalities, ensuring long battery life and consistent performance.

The inclusion of the circuit board 604 enables precise control over the operating parameters of the phototherapy lamp board 526 and the vibrator 606, facilitating intelligent and efficient functionality.

The cosmetic applicator head 100 adopts a dual-surface configuration, with one side dedicated to liquid dispensing and the other designed for phototherapy and massaging functions. The phototherapy side features soft convex structures that provide additional cleansing and massage benefits, improving skincare absorption and relaxation. The liquid dispensing surface ensures precise application of skincare products, optimizing treatment results.

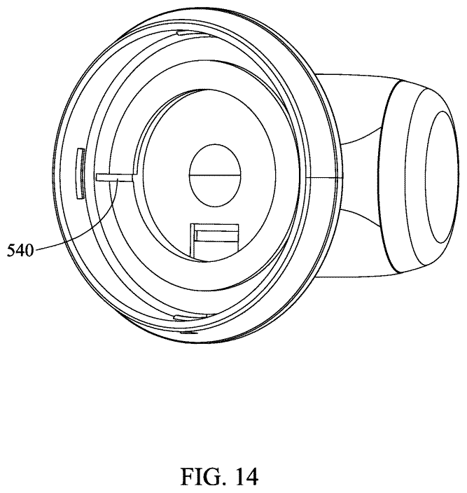

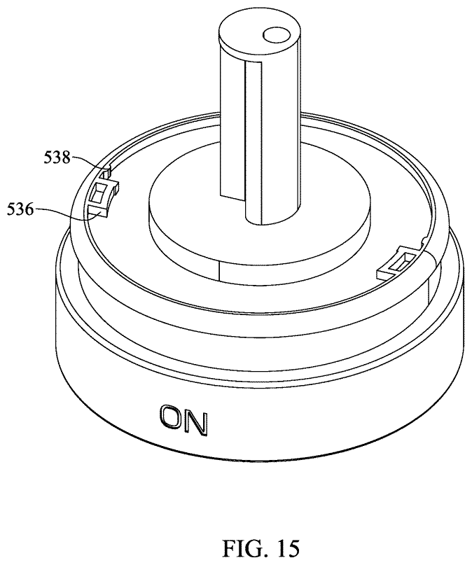

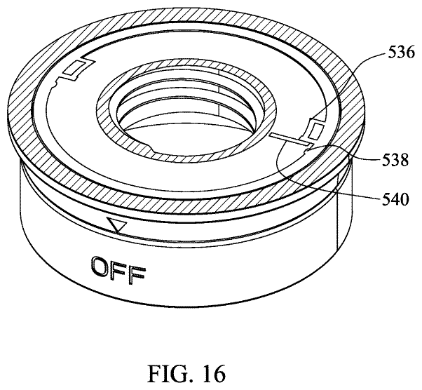

Referring to FIG. 14, FIG. 15 and FIG. 16, the main body 502, and the connecting portion 528 are designed with a limiting protrusion 540 and a first stop protrusion 536, respectively. When the connecting portion 528 is rotated to a preset angle relative to the main body 502, the limiting protrusion 540 engages with the first stop protrusion 536, restricting further movement.

The positioning of the limiting protrusion 540 and the first stop protrusion 536 can be adjusted based on practical requirements, allowing them to be placed on either the main body 502 or the connecting portion 528, as long as they effectively provide resistance and prevent unintended rotation.

Specifically, the connecting portion 528 incorporates a first-stop protrusion 536 and a second-stop protrusion 538, which are spaced apart along the rotation path. The limiting protrusion 540 is capable of passing over the second stop protrusion 538 before engaging with the first stop protrusion 536. Since the first-stop protrusion 536 is larger than the second-stop protrusion 538, the limiting protrusion 540 cannot bypass it. When it reaches the second stop protrusion 538, additional force is required to continue the rotation, providing tactile feedback to the user and indicating proximity to the limit position.

In an embodiment, the connecting portion 528 contains a single set of stop protrusions comprising a first-stop protrusion 536 and a second-stop protrusion 538, allowing it to complete a full rotation while interacting with these stops. In another embodiment, two sets of stop protrusions are incorporated, with each set including a first- stop protrusion 536 and a second-stop protrusion 538. In this configuration, the connecting portion 528 rotates half a turn before engaging the stop protrusions, corresponding to the fully closed and fully open states of the liquid outlet 524. The design ensures stable positioning in either state and minimizes the risk of accidental adjustments.

The number of stop protrusions on the connecting portion 528 is not strictly limited and can be adapted to specific functional needs, as long as they effectively prevent unnecessary rotation and maintain the desired operational state of the cosmetic applicator head 100, thereby reducing the likelihood of misoperation.

Additionally, the liquid outlet 524 and the light-transmitting area 518 are positioned on opposite sides of the main body 502, enabling simultaneous use of the device for both phototherapy and cleansing while preventing liquid contamination of the light-transmitting area 518. The main body 502 features a first side and an opposing second side, with the liquid outlet 524 on the first side and the light-transmitting area 518 on the second side. The distance between these sides gradually decreases in the direction where the connecting portion 528 meets the main body 502, resulting in an inclined arrangement. This ergonomic design ensures that when either side is pressed against the skin, it conforms to facial contours and accommodates natural holding preferences.

Furthermore, the main body 502 includes a housing and a cleaning brush head 520. The housing encloses a storage cavity 506, while the cleaning brush head 520 integrates the liquid outlet 524 and is securely attached to the housing. The cleaning brush head 520 can be conveniently detached using a buckle mechanism, facilitating easy maintenance and cleaning. In alternative embodiments, attachment methods such as screws or magnetic fittings may be utilized.

The cleaning brush head 520 serves to enhance the cleansing process by providing gentle physical friction against the skin. With the integration of the liquid outlet 524, the cleaning brush head 520 ensures that liquid is dispensed effectively during use, improving overall cleansing efficiency.

Referring to FIG. 8 and FIG. 9, the cosmetic applicator head 100 includes a key unit 610, which consists of a key body 612, an electrical connector 614, and a magnet 616. The key body 612 features a mounting hole (not shown in the figures) that extends through both its inner and outer sides. The electrical connector 614 is embedded within this mounting hole, while the magnet 616 is securely fixed to the key body 612. The key body 612 is positioned within the main body 502, and when pressed, it enables the electrical connector 614 to establish contact with the circuit board 604.

The key unit 610 serves as the primary user interface for controlling various functions of the cosmetic applicator head 100, such as activating or deactivating liquid supply, phototherapy, and vibration. Pressing the key body 612 allows the electrical connector 614 to engage with the circuit board 604, triggering the corresponding function.

The electrical connector 614 can take different forms, including a conductive column, a conductive protrusion, or a conductive sheet.

The magnet 616 facilitates magnetic attachment to external power supply structures. For instance, when charging is required, the cosmetic applicator head 100 can magnetically connect to a charger via the magnet 616, allowing the electrical connector 614 to establish an electrical connection with the charging terminal. The number of magnets 616 can vary, and they may be designed in different shapes, such as circular, ring-shaped, or square. To maintain a sleek design and prevent exposure, the magnet 616 is embedded within the key body 612.

Additionally, the utility model includes a liquid supply device that consists of a liquid container (not shown in the figures) and the cosmetic applicator head 100. The liquid container connects to the other end of the liquid channel 534, ensuring a continuous liquid flow. Specifically, the other end of the liquid channel 534 can be designed as an enlarged cavity, accommodating the end of the liquid container. Alternatively, a threaded connection can be implemented on the inner wall of the liquid channel 534, allowing the liquid container to be securely attached.

In an embodiment of the present invention, the cosmetic applicator head comprises one or more stimulation elements to provide additional therapy to the user. The one or more stimulation elements are selected from a group consisting of lasers, heating elements, cooling elements, vibration elements, electrodes, micro-current elements, and a combination thereof. The micro-current element provides micro-current therapy to the user, the vibration element provides massage therapy and the heating element provides heat therapy. The micro-current element or electrodes are either provided on the first surface or the second surface.

In an embodiment of the present invention, the cosmetic applicator head further comprises one or more sensors to sense one or more parameters of the skin. The cosmetic applicator head may communicate one or more parameters of the skin to the external communication device. The one or more sensors upon sensing the one or more parameters of the skin may regulate the operation of one or more stimulation elements.

The foregoing is only an optional embodiment of the present application and is not intended to limit the present application, and any modifications, equivalent substitutions, and improvements, etc., made within the spirit and principles of the present application shall be included in the scope of protection of the present application.

Various modifications to these embodiments are apparent to those skilled in the art, from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A cosmetic applicator head, comprising:
a main body having a mounting end, a smearing end, and a liquid discharge channel extending from the mounting end to the smearing end;
at least one stimulation element to provide one or more therapy to a user;
wherein the mounting end is configured for attachment to a cosmetic container, ensuring fluid communication between the liquid discharge channel and the cosmetic container;
wherein the smearing end comprises a smearing surface through which the liquid discharge channel extends to dispense a cosmetic liquid, a first surface having the at least one stimulation element, and a second surface having multiple convex columns for skin contact.

2. The cosmetic applicator head of claim 1, wherein the first surface comprises a light transmitting area.

3. The cosmetic applicator head of claim 1, wherein the convex columns are designed for massaging, exfoliating, or cleansing the skin.

4. The cosmetic applicator head of claim 1, the at least one stimulation element is selected from a group consisting of light-emitting diodes (LED), lasers, heating elements, cooling elements, vibration elements, electrodes, micro-current elements, and combinations thereof.

5. The cosmetic applicator head of claim 1, further comprising a modular mounting system including: a light-shielding piece positioned within the main body to direct emitted light through the light-transmitting area; and a light-transmitting cover secured over the light-shielding piece.

6. The cosmetic applicator head of claim 5, wherein the light-shielding piece and light-transmitting cover are secured via a snap-fit mechanism or bonding.

7. The cosmetic smear applicator head of claim 1, wherein the main body comprises a shell and a brush head, the shell including a smearing surface and a mounting groove positioned on one side of the smearing surface; wherein the brush head comprises a second surface, a boss, and a plug-in portion extending from the second surface, the plug-in portion being configured to be inserted into the mounting groove for secure attachment.

8. The cosmetic applicator head of claim 1, wherein the first and second surfaces are positioned at an obtuse angle relative to the smearing surface.

9. The cosmetic applicator head of claim 1, wherein the liquid discharge channel comprises:
a first channel within the accessory body; and
a second channel within a liquid guide pipe, with a sealing ring ensuring a secure fit between the two channels.

10. The cosmetic applicator head of claim 1, further comprising an adapter at the mounting end, the adapter is configured to accommodate cosmetic containers of varying sizes.

11. A cosmetic applicator head, comprising:
a main body including a storage cavity and a liquid outlet;
at least one stimulation element to provide therapy to a user;
a connecting portion having a liquid channel, wherein one end of the liquid channel is connected to the liquid outlet and the other end is configured for attachment to a liquid container;
a rotatable coupling between the connecting portion and the main body, enabling alignment adjustment between the liquid channel and the liquid outlet to regulate liquid flow.

12. The cosmetic applicator head of claim 11, wherein the liquid outlet is positioned on a surface that includes brush bristles for enhanced cleansing functionality.

13. The cosmetic applicator head of claim 11, wherein the connecting portion comprises:
a base;
a connecting protrusion extending from the base, wherein the liquid channel passes through the connecting protrusion; and
wherein the connecting protrusion is rotatably matched with the rotating hole in the main body, enabling controlled alignment between the liquid channel and the liquid outlet.

14. The cosmetic applicator head of claim 11, wherein the rotatable coupling allows the liquid channel to be positioned in a fully aligned state for maximum liquid discharge, a fully misaligned state to block liquid flow, and intermediate positions for controlled dispensing.

15. The cosmetic applicator head of claim 11, further comprising a sealing gasket positioned between the connecting component and the main body to prevent liquid leakage.

16. The cosmetic applicator head of claim 11, wherein the main body comprises an outer shell and an inner support, wherein the inner support incorporates a connecting channel that links the liquid channel to the liquid outlet.

17. The cosmetic applicator head of claim 16, wherein the inner support includes a rotating hole accommodating the connecting protrusion, allowing the connecting component to pivot for liquid flow control.

18. The cosmetic applicator head of claim 11, further comprising a limiting protrusion on the main body and a first-stop protrusion on the connecting component to restrict rotational movement within a predetermined range.

19. The cosmetic applicator head of claim 11, wherein the connecting component includes a second stop protrusion positioned along the rotation path to provide tactile feedback when adjusting liquid flow.

20. The cosmetic applicator head of claim 11, wherein the at least one stimulation element is selected from a group consisting of light-emitting diodes (LED), lasers, heating elements, cooling elements, vibration elements, electrodes, micro-current elements, and combinations thereof.

21. The cosmetic applicator head of claim 11, wherein the brush bristles are detachable and secured using a buckle mechanism, screws, or a magnetic attachment.
